(19) United States
(12) Patent Application Publication
(10) Pub. No.: US 2026/0232639 A1
(43) Pub. Date: Aug. 13, 2026

(54) COMPOUNDS FOR USE IN THE TREATMENT OF SCHIZOPHRENIA

(71) Applicant: ONTRACK THERAPEUTICS LIMITED, KENT (GB)

(72) Inventors: Thomas PFEIFFER, London (GB); Alleyn PLOWRIGHT, Peterborough (GB); Man-Ling HO, London (GB)

(21) Appl. No.: 19/156,622

(22) PCT Filed: Feb. 14, 2024
(86) PCT No.: PCT/GB2024/050394

§ 371 Date: Aug. 14, 2025

(30) Foreign Application Priority Data
Feb. 14, 2023 (GB) 2302121.5
Nov. 22, 2023 (GB) 2317870.0

(51) Int. Cl. A61K 31/4188; A61P 25/18; A61P 25/28

(52) U.S. Cl. CPC A61K 31/4188; A61P 25/18; A61P 25/28

(57) ABSTRACT

The present invention relates to the compound 4-(6-oxo-2-(trifluoromethyl)-3,6-dihydrochromeno[7,8-d]imidazol-8-yl)benzonitrile, also known as CF₃CN, referred to herein as the compound of Formula I, for use in the treatment of schizophrenia. Preferably the treatment of schizophrenia is the treatment of one or more of the symptom categories of schizophrenia. More preferably the treatment is for the negative symptoms of schizophrenia. Alternatively, the treatment is for the cognitive symptoms of schizophrenia.

### FIELD OF THE INVENTION

The present invention relates to the compound 4-(6-oxo-2-(trifluoromethyl)-3,6-dihydrochromeno[7,8-d]imidazol-8-yl)benzonitrile, also known as CF₃CN, referred to herein as the compound of Formula I, for use in the treatment of schizophrenia. Preferably the treatment of schizophrenia is the treatment of one or more of the symptom categories of schizophrenia. More preferably the treatment is for the negative symptoms of schizophrenia. Alternatively, the treatment is for the cognitive symptoms of schizophrenia.

### BACKGROUND TO THE INVENTION

The Diagnostic and Statistical Manual of Mental Health (DSM) serves as the universal authority for psychiatric diagnosis. DSM-V, published in 2013, defines the symptoms of schizophrenia into three broad categories: (1) positive symptoms; (2) negative symptoms; and (3) cognitive symptoms.

In general terms the positive symptoms of schizophrenia include hallucinations, delusions, thought disorders and movement disorders. The negative symptoms of schizophrenia include a “flat affect”, a lack of pleasure in everyday life, lack of ability to begin and sustain planned activities, and speaking little, even when forced to interact. Lastly the cognitive symptoms of schizophrenia include poor “executing function”; trouble focusing or paying attention, and problems with working memory.

Treatment of schizophrenia is generally with antipsychotic medications (typical and atypical antipsychotics) which include aripiprazole, asenapine, brexpiprazole, cariprazine, clozapine, iloperidone, lurasidone, olanzapine, paliperidone, quetiapine, risperidone, ziprasidone, chlorpromazine, fluphenazine, haloperidol, and perphenazine.

Other interventions including psychosocial interventions such as therapy, social skills training, vocational rehabilitation and supported employment are also often included in the treatment paradigm.

During periods of severe symptoms patients are often hospitalized to ensure safety for the patient and in cases for patients who do not respond to drug therapy electroconvulsive therapy may be used.

The Positive and Negative Syndrome Scale (PANSS) is an assessment tool used to determine the symptomatology of schizophrenia. Based on two established psychiatric rating systems, the 30-item PANSS was conceived as an operationalized, drug-sensitive instrument that provides balanced representation of positive and negative symptoms and gauges their relationship to one another and to global psychopathology.

In addition the symptoms of schizophrenia relating purely to negative domains can be assessed using the SANS rating scale. This measures the extent of negative symptoms 5 subscales: (i) Affective flattening or blunting; (ii) Alogia; (iii) Avolition/apathy; (iv) Anhedonia/asociality; and (v) Attentional impairment.

The cognitive symptoms of schizophrenia are commonly evident at the time that the first episode of psychosis occurs. Oftentimes the cognitive symptoms are unable to be improved with existing antipsychotic treatments.

In 2006, the FDA indicated that currently available drug treatments for schizophrenia were not satisfactory for the treatment of negative symptoms of the disease (Laughren and Levin, 2006), since this time there have been no medications approved which have solved this issue.

The antipsychotic medications which are approved to treat schizophrenia do not treat specific aspects of the disease in particular they do not address the negative or cognitive symptoms of the disease. As such there is a need for more suitable treatments in particular for these symptoms.

The present application provides data from animal models to demonstrate that the compound of Formula I may be useful in the treatment of schizophrenia. One study demonstrates that the compound of the invention is of use in the treatment is for the cognitive symptoms of schizophrenia. A further study additionally demonstrates that the compound of the invention is of use in the treatment of negative symptoms of schizophrenia.

### BRIEF SUMMARY OF THE DISCLOSURE

In accordance with a first aspect of the present invention there is provided a compound of Formula I for use in the treatment of schizophrenia.

Preferably, the treatment of schizophrenia is the treatment of one or more of the symptom categories of schizophrenia.

More preferably the treatment is for the negative symptoms of schizophrenia. Alternatively, the treatment is for the cognitive symptoms of schizophrenia.

Preferably the compound of Formula I is administered with one or more pharmaceutically acceptable excipients.

Preferably the compound of Formula I is formulated in a dosage form selected from a liquid, a lozenge, a fast-disintegrating tablet, a lyophilized preparation, a film, a spray, an aerosol, a sustained-release tablet or capsule, a modified release tablet or capsule, a tablet, a capsule, a cream, an ointment, or a mucoadhesive.

Preferably the compound of Formula I is administered as a single daily dose.

Alternatively, the compound of Formula I is administered as multiple daily doses. Further still the compound of Formula I is administered two, three, four or five times per day.

Preferably each dose comprises at least 0.001 mg of the compound of Formula I. More preferably each dose comprises between about 0.001 mg and about 500 mg of the compound of Formula I. Alternatively each dose comprises between about 500 mg and about 1000 mg of the compound of Formula I.

In a further embodiment the compound of Formula I is administered with one or more additional drug products.

In accordance with a second aspect of the present invention there is provided a method of treating schizophrenia in a subject in need thereof, the method comprising administering to the subject a therapeutically effective amount of the compound of Formula I.

The compositions, in other embodiments, should provide a dosage of from about 0.0001 mg to about 70 mg of compound per kilogram of body weight per day. Dosage unit forms are prepared to provide from about 0.01 mg, 0.1 mg or 1 mg to about 500 mg, or about 1000 mg, and in some embodiments from about 10 mg to about 500 mg of the active ingredient or a combination of essential ingredients per dosage unit form.

Exemplary doses of a formulation include milligram or microgram amounts of the active compound per kilogram of subject (e.g., from about 1 microgram per kilogram to about 50 milligrams per kilogram, from about 10 micrograms per kilogram to about 30 milligrams per kilogram, from about 100 micrograms per kilogram to about 10 milligrams per kilogram, or from about 100 microgram per kilogram to about 5 milligrams per kilogram).

In certain embodiments, administration of the same formulation provided herein may be repeated and the administrations may be separated by at least 1 day, 2 days, 3 days, 5 days, 10 days, 15 days, 30 days, 45 days, 2 months, 75 days, 3 months, or 6 months.

### BRIEF SUMMARY OF THE DRAWINGS

The present invention is described with reference to the figure listed below:

FIG. 1 details the mean exploration time(s) of two identical objects in the acquisition trial of the NOR test in the acute phase.

FIG. 2 details the mean exploration time(s) of a familiar object and a novel object in the 3 min retention trial of the NOR test in the acute phase.

FIG. 3 details the effect of acute treatment of test compound and risperidone on the discrimination index (DI) in the NOR test.

FIG. 4 details the effect of acute treatment of test compound and risperidone on the total number of line crossings in the acquisition and retention trial of the NOR test.

FIG. 5 details the mean exploration time(s) of two identical objects in the acquisition trial of the NOR test in the 20-day treatment phase.

FIG. 6 details the mean exploration time(s) of a familiar object and a novel object in the 3 min retention trial of the NOR test in the 20-day treatment phase.

FIG. 7 details the effect of 20-day treatment of test compound and risperidone on the discrimination index (DI) in the NOR test.

FIG. 8 details the effect of 20-day treatment of test compound and risperidone on the total number of line crossings in the acquisition and retention trial of the NOR test.

FIG. 9 details the effect of 21-day treatment with test compound and acute treatment of risperidone on sniffing duration in scPCP treated rats.

FIG. 10 details the effect of 21-day treatment with test compound and acute treatment of risperidone on object exploration duration in scPCP treated rats.

FIG. 11 details the effect of 21-day treatment with test compound and acute treatment of risperidone on number of line crossings in scPCP treated rats.

FIG. 12 details the effect of 21-day treatment with test compound (TC) (10 & 30 mg/kg, p.o.) on parvalbumin interneuron cell density (cells/mm2) in the subregions of the prefrontal cortex. Data are expressed as mean±s.e.m (n=7-9 per group). *=p<0.05, **=p<0.01. Cg1=cingulate cortex′ PrL=prelimbic cortex, IL=infralimbic cortex.

FIG. 13 details the effect of 21-day treatment with test compound (TC) (10 & 30 mg/kg, p.o.) on parvalbumin interneuron cell density (cells/mm2) in the prefrontal cortex. Data are expressed as mean±s.e.m (n=7-9 per group). *=p<0.05, **=p<0.01 vs scPCP.

FIG. 14 details the effect of 21-day treatment with test compound (TC) (10 & 30 mg/kg, p.o.) on parvalbumin interneuron cell density (cells/mm2) in the subregions of the dorsal hippocampus. Data are expressed as mean±s.e.m (n=7-9 per group). CA1=Cornu Ammonis 1, CA2/3=Cornu Ammonis 2/3, DG=dentate gyrus.

FIG. 15 details the effect of 21-day treatment with test compound (TC) (10 & 30 mg/kg, p.o.) on parvalbumin interneuron cell density (cells/mm2) in the dorsal hippocampus. Data are expressed as mean±s.e.m (n=7-9 per group).

FIG. 16 details the effect of 21-day treatment with test compound (TC) (10 & 30 mg/kg, p.o.) on BDNF levels (pg/ml) in the prefrontal cortex. Data are expressed as mean±s.e.m (n=7-9 per group).

FIG. 17 details the effect of 21-day treatment with test compound (TC) (10 & 30 mg/kg, p.o.) on BDNF levels (pg/ml) in the dorsal hippocampus. Data are expressed as mean±s.e.m (n=9-11 per group).

FIG. 18 details the effect of 21-day treatment with test compound (TC) (10 & 30 mg/kg, p.o.) on SNAP25 in the prefrontal cortex. Data are expressed as mean±s.e.m (n=6-8 per group). *=p<0.05 vs scPCP.

FIG. 19 details the effect of 21-day treatment with test compound (TC) (10 & 30 mg/kg, p.o.) on PSD95 in the prefrontal cortex. Data are expressed as mean±s.e.m (n=7-9 per group).

FIG. 20 details the effect of 21-day treatment with test compound on SCPCP induced cognitive deficits in the set-shifting task.

FIG. 21 details the effect of 21-day treatment with test compound on synaptic markers PSD95 and SNAP25 in prefrontal cortex.

FIG. 22 details the effect of 21-day treatment with test compound on BDNF concentration in the dorsal hippocampus.

### DEFINITIONS

Various definitions are made throughout this document. Most words have the meaning that would be attributed to those words by one skilled in the art. Words specifically defined either below or elsewhere in this document have the meaning provided in the context of the present invention as a whole and as typically understood by those skilled in the art.

“Subject,” “individual” or “patient” is used interchangeably herein and refers to a vertebrate, preferably a mammal. Mammals include, but are not limited to, murines, rodents, simians, humans, farm animals, sport animals and pets.

“Treating” or “treatment” of any disease or disorder refers, in some embodiments, to ameliorating the disease or disorder (i.e., arresting or reducing the development of the disease or at least one of the clinical symptoms thereof). Treatment may also be considered to include preemptive or prophylactic administration to ameliorate, arrest or prevent the development of the disease or at least one of the clinical symptoms. Treatment can also refer to the lessening of the severity and/or the duration of one or more symptoms of a disease or disorder. In a further feature, the treatment rendered has lower potential for long term side effects over multiple years. In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter) or both. In yet other embodiments, “treating” or “treatment” refers to delaying the onset of the disease or disorder.

“Therapeutically effective amount” means the amount of a compound that, when administered to a patient for treating a disease, is sufficient to affect such treatment for the disease. The “therapeutically effective amount” will vary depending on the compound, the disease and its severity and the age, weight, adsorption, distribution, metabolism and excretion etc., of the patient to be treated.

“Vehicle” refers to a diluent, excipient or carrier with which a compound is administered to a subject. In some embodiments, the vehicle is pharmaceutically acceptable.

“Active ingredient” or “Active pharmaceutical ingredient” or “API” refers to the compound of the invention.

“4-(6-oxo-2-(trifluoromethyl)-3,6-dihydrochromeno[7,8-d]imidazol-8-yl)benzonitrile”, also known as “CF₃CN”, referred to herein as the “compound of Formula I” has a SMILES code N #CC1═CC═C(C2═CC(C3═CC═C4C(N═C(C(F)(F)F)N4)═C3O2)═O)C═C1 and the structure defined below:

Compound of Formula I

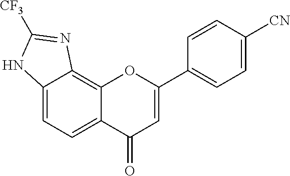 [SMILES: N#Cc1ccc(-c2cc(=O)c3ccc4[nH]c(C(F)(F)F)nc4c3o2)cc1]

### DETAILED DESCRIPTION OF THE INVENTION

The compound tropoflavin, also known as 7,8-dihydroxyflavone (7,8-DHF), is a naturally occurring flavone found in Godmania aesculifolio, Tridax procumbent and primula tree leaves. It is known to act as a potent and selective agonist of tropomyosin receptor kinase B (TrkB), which is the main signaling receptor of neurotrophin brain-derived neurotrophic factor (BDNF).

Tropoflavin has been shown to have therapeutic efficacy in several animal models including depression, Alzheimer's disease, cognitive deficits in schizophrenia, Parkinson's disease, Huntington's disease, amyotrophic lateral sclerosis, traumatic brain injury, cerebral ischemia, fragile X syndrome and Rett syndrome.

A derivative of tropoflavin, 4-(6-oxo-2-(trifluoromethyl)-3,6-dihydrochromeno[7,8-d]imidazol-8-yl)benzonitrile, also known as CF₃CN, referred to herein as the compound of Formula I, has been shown to be useful in the treatment of schizophrenia.

The Examples below describes the effect of the compound of Formula I in animal models which demonstrate the compound's ability to treat the symptoms of schizophrenia.

The novel object recognition (NOR) test is a two-trial cognitive paradigm that assesses visual recognition memory. A recognition memory task allows the comparison between presented stimuli and previously stored information. Recognition memory is disturbed in a range of human disorders and NOR is widely used in rodents for investigating deficits in a variety of animal models of human conditions where cognition is impaired.

The social interaction test is a putative animal model for assessing social withdrawal, this is an aspect of the avolition domain of negative symptoms in CNS disorders such as schizophrenia. The social interaction test is a 10 minute, one-trial task that assesses the behavioural interaction of a test rat with an un-familiar, weight matched conspecific. This test detects behavioural changes in social behaviour and is widely used in rodents for investigating deficits in a variety of animal models of human conditions where social impairments are observed (e.g., aspects of the negative symptomatology associated with schizophrenia).

### Example 1: Effect of Test Compound on Sub-Chronic Pcp-Induced Deficits in Recognition Memory Using the Novel Object Recognition (NOR) Paradigm in Rats

The novel object recognition test (NOR) test is a two-trial cognitive paradigm that assesses visual recognition memory. A recognition memory task allows the comparison between presented stimuli and previously stored information. Recognition memory is disturbed in a range of human disorders and NOR is widely used in rodents for investigating deficits in a variety of animal models of human conditions where cognition is impaired.

Following initial habituation to the empty test arena, the test consists of two trials. In the first trial, the rats are exposed to two identical objects in an open arena (acquisition phase). In the second trial, following an interval (1 min-6 h, depending on the type of memory and brain region of interest), rats are exposed to two dissimilar objects, one familiar object from the first trial and one novel object (retention phase). Object recognition can be measured as the difference in time spent exploring the familiar and the novel object. Behaviour is recorded and scored using an NOR scoring timer by a trained experimenter who is blind to the treatment groups.

Total object exploration time (defined as the duration of time animals spent licking, sniffing, or touching the object but not including time spent standing or sitting on or leaning against the object) is recorded for each of the familiar and novel objects in the acquisition and retention trials. Discrimination index (defined as the difference in time spent exploring the novel and the familiar objects divided by total time spent exploring both objects) and locomotor activity (defined as movement, measured by the number of lines crossed in both trials) are also calculated. Rats have been shown to spend more time exploring the novel object in the retention phase.

Treatment of the rats with subchronic Phencyclidine (scPCP) induces a selective, long lasting and robust deficit in the NOR paradigm. The present study was conducted to evaluate the ability of the compound of Formula I to reverse scPCP deficit in the NOR paradigm. The effects of scPCP in this paradigm may represent a selective deficit in visual recognition memory which is known to be impaired in schizophrenia and as such test compounds which are shown to be effective in this model may be of use in the treatment of cognitive symptoms of schizophrenia.

### Materials and Methods

### Drug Preparation and Administration:

Compound of Formula I (5% DMSO in 0.5 Methylcellulose 400cp) was given at 10 & 30 mg/kg p.o. in a dose volume of 5 ml/kg. This was made fresh daily and constantly stirred prior to dosing. On behavioural days (D1 & D20) Compound of Formula I was administered p.o. 120 mins prior to behavioural testing.

### Animals-Acclimatisation and Housing Conditions:

Rats were housed in groups of 4-5 under standard laboratory conditions, under a 12 hr light:dark cycle (lights on at 07:00 hr).

Testing was carried out in the light phase. Animals had free access to food and water, except during the behavioural testing procedures.

Animals were weighed regularly throughout the experiment.

All procedures were carried out in accordance with the Animal Scientific Procedures Act (UK, 1986) and are approved by the University of Manchester's AWERB (Animal Welfare and Ethical Review Body).

### Novel Object Recognition (NOR) Protocol:

A total of 66 female Lister Hooded rats were used for the NOR study. 56 rats were treated sub-chronically with PCP and 10 with vehicle.

The PCP injection regimen is 2 mg/kg i.p. twice daily for 7 days followed by at least a 7-day drug-free period. This regimen has been found to produce robust and long lasting behavioural and pathological deficits in this paradigm (Neill et al., 2010; Cadinu et al, 2017). Following the scPCP dosing regimen and prior to behaviour testing, rats were treated as follows:

1. Acute Treatment with Test Compound:

Rats (n=36) were treated acutely with Compound of Formula I (API) at two doses (10 mg/kg (n=18) & 30 mg/kg (n=18), p.o., 120 mins prior to testing on day 1), risperidone (0.1 mg/kg, i.p., 60 min prior to testing) or vehicle (5% DMSO in 0.5 Methylcellulose 400cp, p.o., 120 mins prior to testing on day 1). Brains (n=3) from each of the API groups were taken immediately following behavioural testing for PK analysis.

2. 20-Day Treatment with Test Compound:

Rats (n=30) were dosed for 20-days with Compound of Formula I (API) at two doses (10 mg/kg (n=15) & 30 mg/kg (n=15), p.o., 120 mins prior to testing on day 20), risperidone (0.1 mg/kg, i.p., 60 min prior to testing) or vehicle (5% DMSO in 0.5 Methylcellulose 400cp, p.o., 120 mins prior to testing on day 20). Brains (n=3) from each of the API groups were taken immediately following behavioural testing for PK analysis.

### Statistical Analysis:

All data are expressed as mean±s.e.m. (standard error of mean). Exploration time data from NOR in the acquisition and retention phases were analysed separately via repeated measures GLM with factors of drug and exploration time of the 2 objects (2 identical objects in the acquisition phase, and novel and familiar objects in the retention phase). Discrimination index (DI) and locomotor activity data (total number of line crossings in both phases) were analysed via one-way ANOVA. Post-hoc analysis was conducted following a significant one-way ANOVA by the least significant difference (LSD) test (for locomotor activity and DI scores).

Results—Acute Treatment with Test Compound:

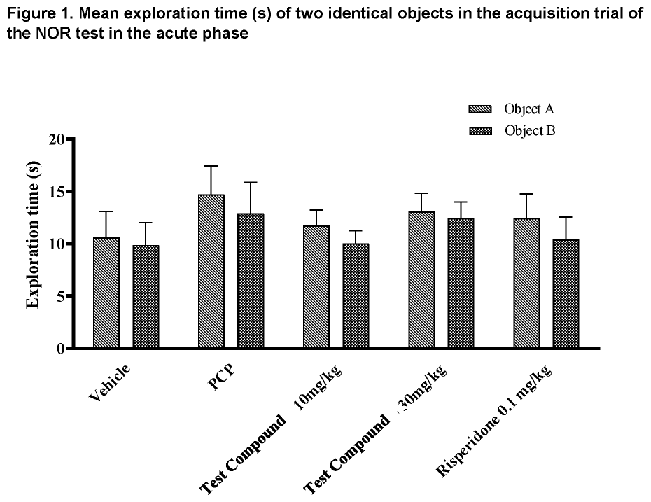

The effect of acute treatment with Compound of Formula I (API) at doses of 10 & 30 mg/kg, p.o. and the positive control risperidone (0.1 mg/kg, i.p.) on the total object exploration time(s) in the acquisition and retention trial are described in Table 1 below and FIGS. 1 to 4.

TABLE 1
Total time spent by rats during the two parts of the NOR trial

| | Acquisition | Retention |
|---|---|---|
| | total exploration | total exploration |
| Treatment | time (s) | time (s) |
| Vehicle + Vehicle | 20.46 ± 3.79 | 23.05 ± 3.08 |
| scPCP + Vehicle | 27.61 ± 5.56 | 27.97 ± 3.18 |
| scPCP + API 10 mg/kg | 21.75 ± 2.46 | 22.55 ± 1.60 |
| scPCP + API 30 mg/kg | 25.49 ± 3.04 | 26.18 ± 2.11 |
| Risperidone 0.1 mg/kg | 22.81 ± 4.03 | 23.10 ± 2.92 |

As can be seen there was no difference in the amount of time spent exploring two identical objects in the acquisition part of the trial (FIG. 1) by any of the treatment groups as was expected.

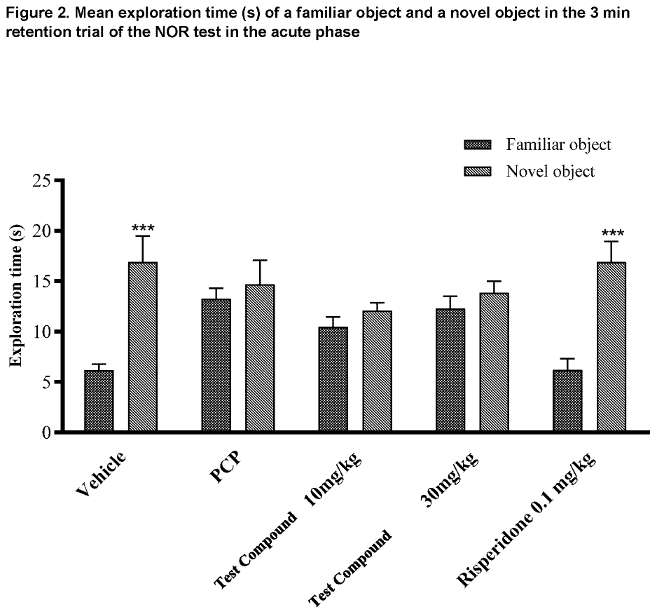

The positive control and the vehicle treated animals showed a statistically significant increase in the amount of time spent exploring the novel object in the retention part of the trial (FIG. 2). The animals not treated (PCP only) and the animals treated acutely with the compound of Formula I were not shown to have a difference in the amount of time exploring the novel or familiar object.

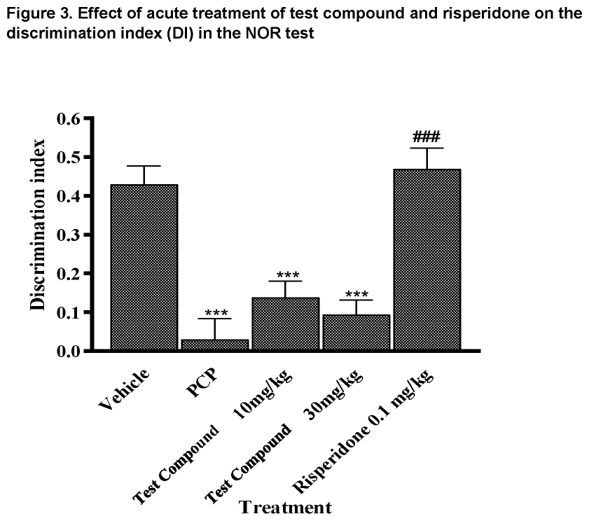

As would be expected from the results shown in FIG. 3, both the vehicle and risperidone treated animals had a higher discrimination index (DI) than those without treatment and those treated with test compound, (FIG. 3).

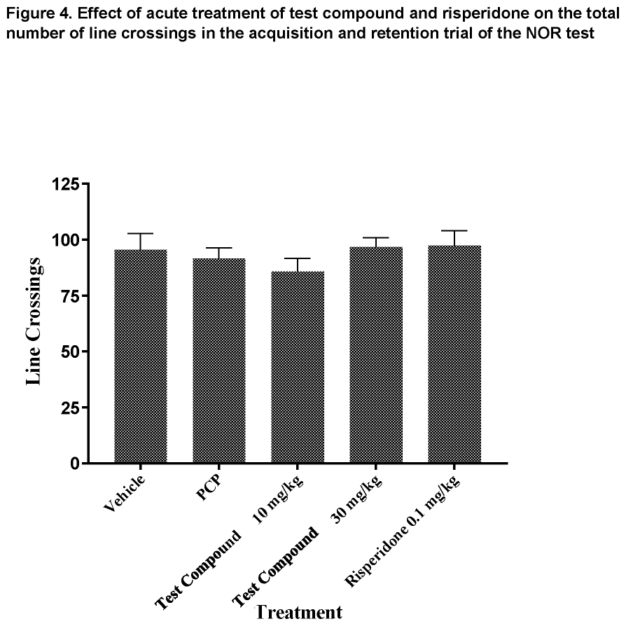

FIG. 4 demonstrates that there was no impact on any of the treatments on locomotor activity as there was no difference in the number of line crossings made within the treatment groups.

Results—20-Day Treatment with Test Compound:

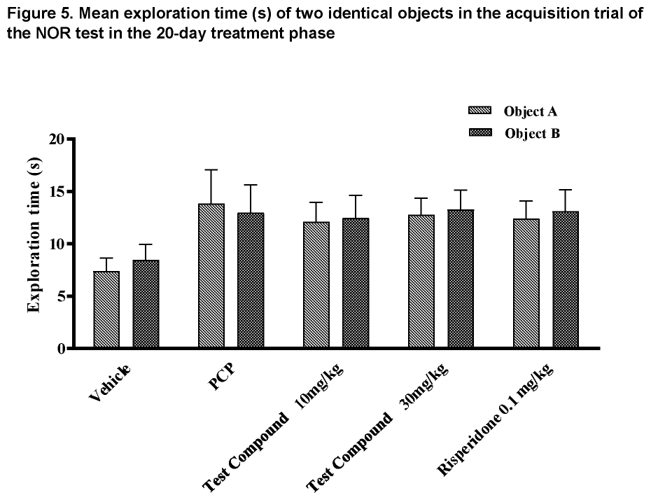

The effect of once-daily treatment with Compound of Formula I (API) at doses of 10 & 30 mg/kg, p.o. for 20 days and acute treatment with the positive control risperidone (0.1 mg/kg, i.p.) on the total object exploration time(s) in the acquisition and retention trial are described in Table 2 below and FIGS. 5 to 8.

TABLE 2
Total time spent by rats during the two parts of the NOR trial

| | Acquisition | Retention |
|---|---|---|
| | total exploration | total exploration |
| Treatment | time (s) | time (s) |
| Vehicle + Vehicle | 15.9 ± 2.6 | 16.0 ± 1.7 |
| scPCP + Vehicle | 26.8 ± 5.3 | 16.1 ± 1.2 |
| scPCP + OT-003 10 mg/kg | 24.6 ± 3.4 | 19.0 ± 1.4 |
| scPCP + OT-003 30 mg/kg | 26.1 ± 3.1 | 20.8 ± 1.2* |
| Risperidone 0.1 mg/kg | 25.5 ± 3.4 | 17.8 ± 1.1 |

*P < 0.0.5; significant increase in exploration compared to scPCP.

As can be seen there was no difference in the amount of time spent exploring two identical objects in the acquisition part of the trial (FIG. 5) by any of the treatment groups as was expected.

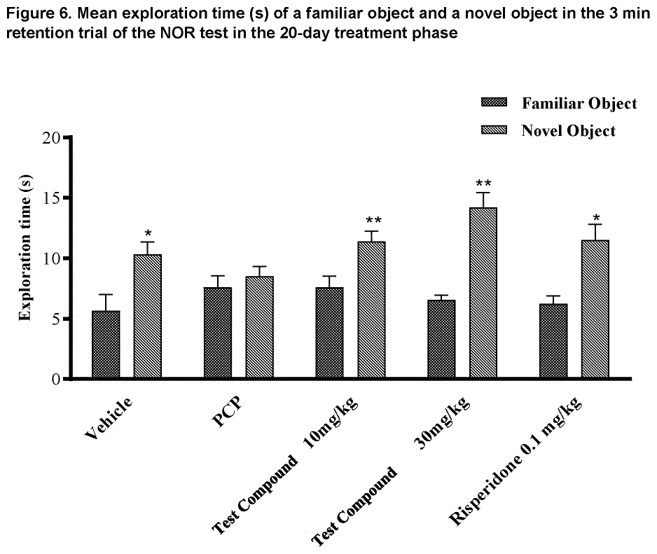

The positive control and the vehicle treated animals showed a statistically significant increase (P<0.05) in the amount of time spent exploring the novel object in the retention part of the trial (FIG. 6). The animals not treated (PCP only) were not shown to have a difference in the amount of time exploring the novel or familiar object.

FIG. 6 further demonstrates a dose dependent statistically significant effect in the amount of time spent exploring the familiar and novel object in the animals treated with the compound of Formula I (**P<0.01). This effect was greater than that seen with the positive control risperidone.

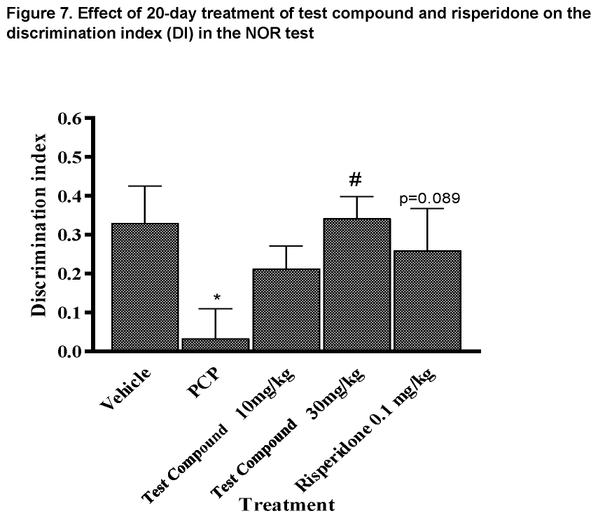

FIG. 7 details the discrimination index (DI) for all the treatment groups. It can be seen that the effect of 20-day treatment of the compound of Formula I at 30 mg/kg produced a statistically significant increase in the DI (P<0.015) compared to the animals without treatment.

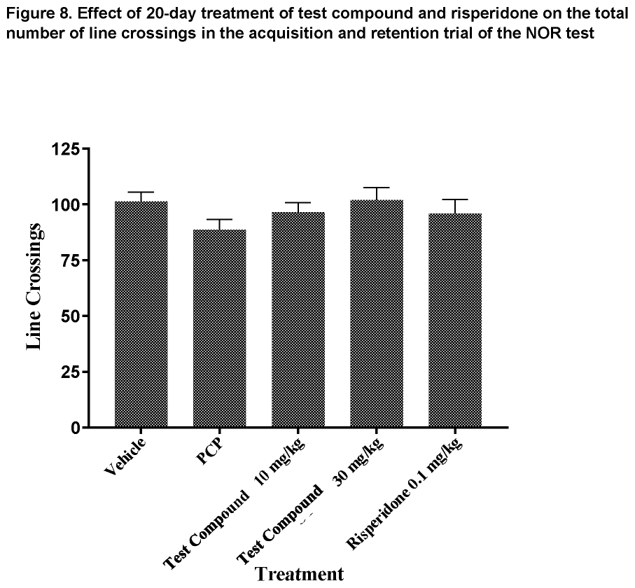

FIG. 8 demonstrates that there was no impact on any of the treatments on locomotor activity as there was no difference in the number of line crossings made within the treatment groups.

### Conclusion

The NOR test has been previously demonstrated to identify compounds that may be useful in the treatment of visual recognition memory which is known to be impaired in schizophrenia. The data presented in Example 1 demonstrates that the positive control risperidone, dosed acutely, was able to rescue the deficits induced by scPCP in the rat, indicative that the test was robust.

Importantly, the data additionally demonstrates that dosing animals for a 20-day period with the compound of Formula I is able to rescue the scPCP deficit in a dose dependent manner.

Such a finding provides evidence that the compound of Formula I might be suitable for use in the treatment of the cognitive symptoms of schizophrenia.

### Example 2: Effect of Test Compound on Social Interaction in Sub-Chronic PCP-Induced Deficits in Rats

The social interaction test is a putative animal model for assessing social withdrawal, this is an aspect of the avolition domain of negative symptoms in CNS disorders such as schizophrenia. The social interaction (SI) test is a 10 minute, one-trial task that assesses the behavioural interaction of a test rat with an un-familiar, weight matched conspecific. This test detects behavioural changes in social behaviour and is widely used in rodents for investigating deficits in a variety of animal models of human conditions where social impairments are observed (e.g. aspects of the negative symptomatology associated with schizophrenia).

Following initial habituation to the empty test arena, the test consists of one trial. In this trial, the test animal (i.e. scVeh, scPCP, scPCP+treatment) is put into the arena with an unfamiliar weight matched conspecific.

The main behaviours scored in this test are:
1. Sniffing behaviour
2. Investigation of an inanimate object (control)
3. Line crossings to measure locomotor activity

Behaviour is recorded and scored by a trained experimenter who is blind to the treatment groups.

The scPCP regimen has been demonstrated to produce a selective, long lasting and robust deficit in this task. Using the scPCP model in this test, robust reductions in pro-social behaviour (i.e. sniffing) with no difference in exploration of the unfamiliar inanimate object are observed.

### Materials and Methods

A total of 54 female Lister Hooded rats were used for the SI study. 44 rats will be treated sub-chronically with PCP and 10 with vehicle. The PCP injection regimen is 2 mg/kg i.p. twice daily for 7 days followed by at least a 7-day drug-free period. This regimen has been found to produce robust and long lasting behavioural and pathological deficits in this paradigm (Neill et al., 2010; Cadinu et al., 2017).

Following the scPCP dosing regimen as detailed in Example 1 above, and prior to behaviour testing, rats were treated for 21 days with test compound.

Rats (n=24) were dosed for 21-days with test compound at two doses (10 mg/kg (n=12) & 30 mg/kg (n=12), p.o., 120 mins prior to testing on day 21), the positive control used was risperidone (0.1 mg/kg, i.p., 60 min prior to testing) or vehicle (5% DMSO in 0.5 Methylcellulose 400cp, p.o., 120 mins prior to testing on day 21). Brains (n=3) from each of the test article groups were taken immediately following behavioural testing for PK analysis.

### Statistical Analysis:

All data will be expressed as mean±s.e.m (n=10-12 per group) and analysed by a one-way ANOVA with further analysis by Dunnett post hoc correction analysis.

### Results

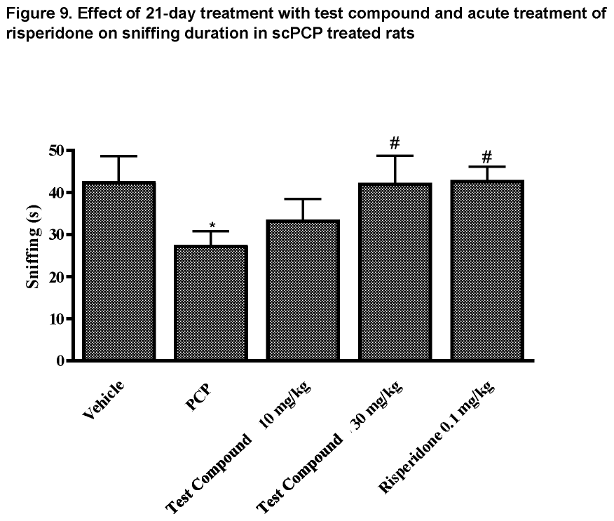

FIGS. 9 to 11 detail the results produced in the scPCP treated rats after 21 days of treatment with the compound of Formula I.

FIG. 9 shows that treatment with PCP significantly reduces the amount of time spent sniffing demonstrating that the treatment with PCP produces a social impairment in the test animals.

FIG. 9 goes on to detail that the compound of Formula I was able to rescue the social impairment deficit in the scPCP treated rats in a dose dependent manner by increasing the amount of time sniffing. The higher dose of 30 mg/kg was shown to be statistically significant (P<0.05).

In addition, the positive control was shown to increase the amount of time the rat spent sniffing to a similar level as that observed by the higher dose of test article.

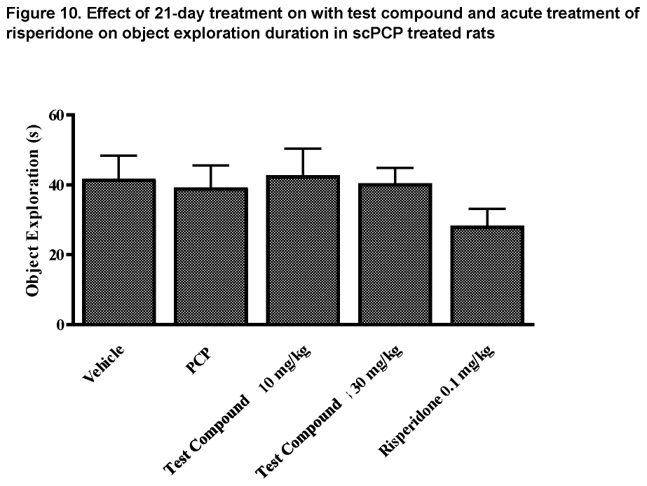

FIG. 10 shows that both the vehicle and PCP treated animals spent a similar amount of time exploring the inanimate object demonstrating the robustness of the test. In addition, there were no differences between the test article and the positive control as would be expected.

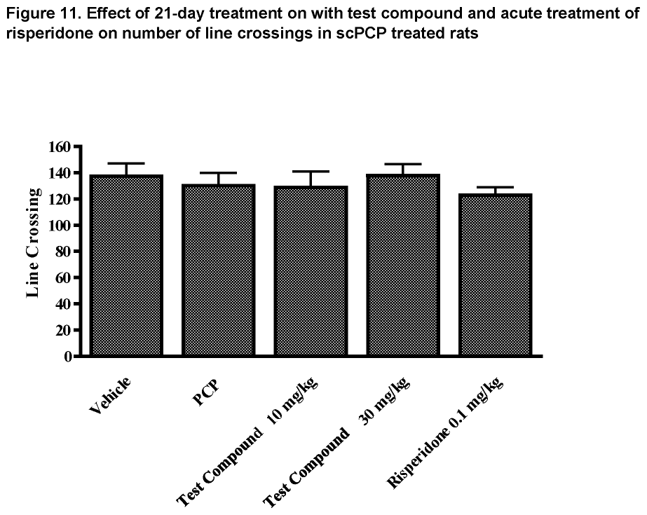

Lastly, there was no difference in line crossings showing that the treatment had no effect on locomotor activity (FIG. 11).

### Conclusion

The Social Interaction test has been previously demonstrated to identify compounds that may be useful in the treatment of negative symptoms in CNS disorders. The data presented in Example 2 demonstrates that the positive control risperidone, dosed acutely, was able to rescue the social impairment deficits induced by scPCP in the rat, indicative that the test was robust.

Importantly, the data additionally demonstrates that dosing animals for a 21-day period with the compound of Formula I is able to rescue the social impairment in a dose dependent manner.

Such a finding provides evidence that the compound of Formula I might be suitable for use in the treatment of the negative symptoms of schizophrenia.

### Example 3: Investigating the Effect of 21-Day Treatment with Test Compound on Pathological Markers in the Prefrontal Cortex (Parvalbumin Interneurons, BDNF, SNAP25 & PSD95) and Dorsal Hippocampus (Parvalbumin Interneurons & BDNF) OF scPCP-Treated Rats

This example investigated the effects of 21-day treatment with the compound of Formula I (10 & 30 mg/kg, p.o.) on post-mortem markers (BDNF, PVIs and synaptic markers) in two different brain regions of relevance to the cognitive and negative symptoms of schizophrenia.

### Materials and Methods

### Brain Collection

Immediately following the behavioural analysis in previous examples, the brains were taken and prepared for post-mortem analysis. Animals were culled by increasing concentration of carbon dioxide and death was confirmed by cervical dislocation. Brains were removed, frozen on dry ice and stored at −80° C. until analysis.

½ brains (one hemisphere) were randomly selected and processed for parvalbumin immunohistochemistry (IHC), quantified in the prefrontal cortex and dorsal hippocampus.

Remaining ½ brains (remaining hemispheres) were processed for protein analysis of BDNF using ELISA (quantified in the prefrontal cortex and the dorsal hippocampus) and synaptic markers using Simple Western (quantified in the prefrontal cortex).

### Immunohistochemistry

### Staining:

Sections (30 μm thickness) were washed in 1× PBS three times for five minutes each. Sections underwent heat-induced antigen retrieval in sodium citrate buffer (0.294% (w/v) sodium citrate, 0.07% (v/v) tween-20, 500 ml distilled water, pH adjusted to 6.0) for 30 minutes at 80° C. Unless otherwise stated, samples were washed twice in 1× PBS between each step.

Parvalbumin (PV) sections were processed for light microscopy. Samples were incubated in hydrogen peroxide solution (1.5% H2O2, Sigma; 0.4% Triton x-100, Sigma; 10% methanol, 88.1% 1× PBS) for 30 minutes at room temperature (RT). Samples were incubated in protein block (5% normal horse serum, Vector Laboratories; 0.6% triton x-100, Sigma; 94.4% 1× PBS) for one hour. Then, without washing, incubated in parvalbumin (1:5000; 24 hours; 235, Swant) at 4° C. On day 2, samples were incubated with the secondary antibody (PV: horse anti-mouse; 1:200; BA-2000-15, Vector Laboratories) for 2 hours. Sections were incubated with the VECTASTAIN Elite ABC-HRP kit (PK-4000, Vector Laboratories) for 45 minutes and visualised with DAB substrate kit (SK-4100, Vector Laboratories). When a sufficient colour change had been observed, samples were moved into distilled water to stop the peroxidase reaction. Sections were mounted onto Superfrost slides and assigned randomised codes. Slides were then dehydrated in increasing ethanol concentrations (70%, 90% and 100%, 5 minutes each) and washed in Histoclear (National Diagnostics) for 5 minutes. Coverslips were applied with DPX mounting media (Sigma).

### Image Analysis:

Images were acquired on a 3D-Histech Pannoramic-250 microscope slide-scanner using a 20×/0.80 Plan Apochromat objective (Zeiss). Snapshots of the slide scans were taken using the CaseViewer software (3D-Histech). For PV images, the region of interest was delineated, and the number of cell detections were manually counted within the CaseViewer software. Density was calculated by dividing the number of positive cell detections by the area of the region of interest.

### Protein Analysis

### Homogenisation:

Prefrontal Cortex and dorsal hippocampus were dissected from the frozen brain and homogenised on ice in a 10-fold volume (mg:μL) of buffer (10 mM Trizma base, Sigma-Aldrich; 320 μM sucrose, Sigma-Aldrich; 2 mM EDTA, Sigma-Aldrich; and protease- and phosphatase-inhibitor cocktails, Sigma-Aldrich) buffered to a pH of 7.4. Phenylmethylsulfonyl fluoride (PMSF) and sodium orthovanadate were added to the solution at a 1% (v/v) concentration. Samples were centrifuged at 800×g for 15 minutes at 4° C. The supernatant was collected and centrifuged at 11,700×g for 20 minutes, and the resulting supernatant was collected and stored at −20° C. until analysis.

### Bradford Assay:

Protein concentrations of each sample were calculated using a Bradford Assay. Samples were diluted into protein assay dye reagent concentrate (BioRad) and compared to bovine serum albumin (Sigma) of known concentration (1.0-0.1 mg/ml protein). A standard curve was created using a simple linear regression. Standards and samples were run in triplicate. An outlying value from a triplicate was removed if the CV value was greater than 10%.

### ELISA (Brain Derived Neurotrophic Factor):

Quantitative measurement of BDNF in brain tissue was conducted using a Rat Brain Derived Neurotrophic Factor (BDNF) ELISA Kit (Cat #EKU02787-96T, BIOMATIK). Briefly samples were diluted to 2.5 mg/mL protein. An 8-point, 2-fold standard curve was created (31.25-1000 μg/mL), including a diluent-only control. The standards and samples were pipetted into the pre-coated 96-well plate and incubated for 1 hour at 37° C. The samples were removed from each well without washing. Next, detection reagent A was pipetted into the plate and incubated for one hour at 37° C. The samples were washed eight times with wash buffer, and the remaining wash solution was removed from the plate by inverting the plate onto absorbent paper. Then, detection reagent B was added and incubated for 30 minutes at 37° C. The wash step was repeated. Finally, the substrate solution was added and developed in the dark. Once the stop solution was added, the plate was read on a microplate reader at 450 nm.

Samples for BDNF analysis in the total dorsal hippocampus were run across two separate ELISA plates. A subset of samples from each group were ran on both plates to investigate inter-assay variability. Data confirmed that this was <10% (Table 3).

TABLE 3
BDNF levels (pg/ml) across different ELISA plates.
S1-S11 = Samples across all four groups.

| Plate Repeats | Plate A | Plate B | % MATCH |
|---|---|---|---|
| S1 | 369.87 | 346.47 | 93.67 |
| S2 | 352.16 | 339.91 | 96.51 |
| S3 | 336.66 | 316.26 | 93.94 |
| S4 | 316.08 | 317.58 | 100.46 |
| S5 | 316.08 | 291.28 | 92.15 |
| S6 | 314.27 | 303.10 | 96.44 |
| S7 | 377.54 | 404.24 | 107.07 |
| S8 | 321.82 | 297.86 | 92.55 |
| S9 | 360.39 | 397.67 | 110.34 |
| S10 | 282.02 | 264.94 | 93.94 |
| S11 | 377.98 | 346.99 | 91.80 |
| MEAN | 338.62 | 329.66 | 97.17 |
| N | 11 | 11 | 11 |
| SEM | 9.44 | 13 | 1.89 |

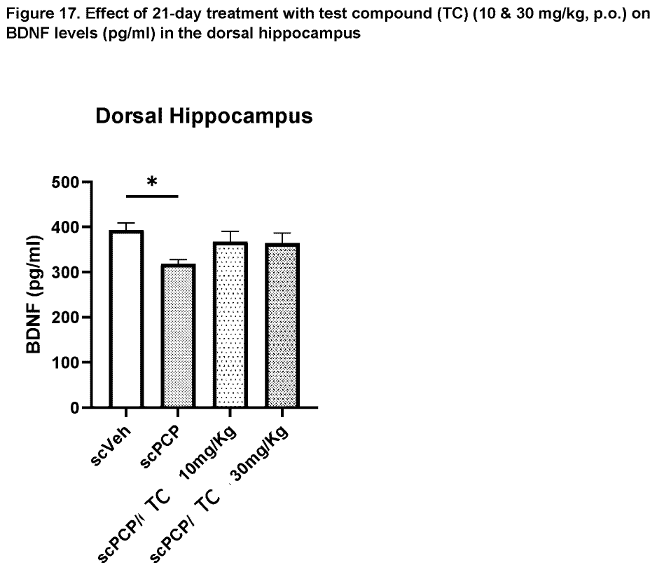

Samples from both plates were combined for the final analysis of BDNF in the total dorsal hippocampus (FIG. 17). Specifically, plate A values were used where available, while for samples with only plate B values, these were used instead.

### Simple Western (Synaptic Markers)

A WES plate was prepared following the provided instructions (SM-W004, ProteinSimple). Diluted samples were mixed with dithiothreitol and fluorescent master mix in an 8:1:1 ratio. Samples were denatured at 95° C. for five minutes. The samples, antibody diluent, primary antibody, secondary antibody and luminol-peroxide mix were loaded into appropriate wells. A biotinylated ladder, antibody diluent, streptavidin-HRP and luminol peroxide were also loaded. After brief centrifugation of the plate, wash buffer was added. The plate was then placed into the WES machine, whereby the load separation matrix is aspirated, and stacking matrix drawn up into each capillary. The ladder and samples were then loaded and lowered into running buffer. An electrical current was applied to the samples, and they were separated based on molecular weight. At the end of the separation, samples were immobilised by UV light, removing the lysis buffer and non-protein components. Then, the capillaries draw up the blocking solution, primary and secondary antibodies, flanked by wash steps. Finally, the chemiluminescent substrate was added into the capillaries, and the camera detected the levels of chemiluminescence at 9 different timepoints (1-512 seconds). The software then calculated the high dynamic range (HDR) of the peaks, which is calculated from the individual exposures and provides the largest dynamic range of the signal with the lowest background. The HDR peak is then used to calculate the area under the curve, which is analogous to the quantity of protein in the sample. Antibodies and dilutions used are shown in Table 4 below.

TABLE 4
Antibody details for simple western analysis.

| | | | Protein | |
|---|---|---|---|---|
| | Product | | concentration | Antibody |
| Target | code | Supplier | (mg/mL) | dilution |
| SNAP25 | Ab5666 | Abcam | 0.4 | 1:50 |
| PSD95 | Ab2723 | Abcam | 0.4 | 1:50 |

To normalise the proteins of interest we use a total protein assay provided by protein simple (DM-TP01). The area under the curve of the protein of interest is divided by the area under the curve for the total protein to find the normalised relative protein levels.

### Statistical Analysis

Parvalbumin density in the Prefrontal Cortex and Dorsal Hippocampus were analysed using a repeated measure two-way ANOVA with region and treatment as fixed factors. This was followed by Fisher's LSD post hoc test to find the difference between the treatment groups within the respective subregions.

BDNF and synaptic markers were analysed using a one-way ANOVA followed by planned post hoc analysis to investigate differences between treatment groups for Prefrontal Cortex (BDNF & Synaptic markers) and Dorsal Hippocampus (BDNF) respectively.

### Results

Effect of 21-Day Treatment with Test Article on Parvalbumin Interneurons in the Prefrontal Cortex

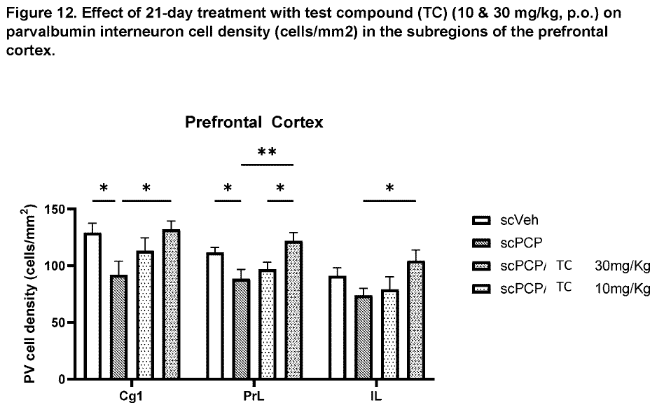

In the prefrontal cortex, there was a significant main effect of treatment and region, with no interaction. In both the cingulate and prelimbic cortex there was a significant reduction in PV cell density in scPCP animals (both p<0.05 vs scVeh). Chronic treatment with the compound of Formula I at 30 mg/kg significantly increased PV cell density in scPCP-treated rats across all three regions (Cg1 & IL-p<0.05; PrL-p<0.01) (FIG. 12).

A one-way ANOVA found a significant main effect of treatment in the total prefrontal cortex (F (3,26)=3.053; p=0.0462). Planned post hoc analysis found a reduction in PV cell density in scPCP animals (p<0.05 vs scVeh).

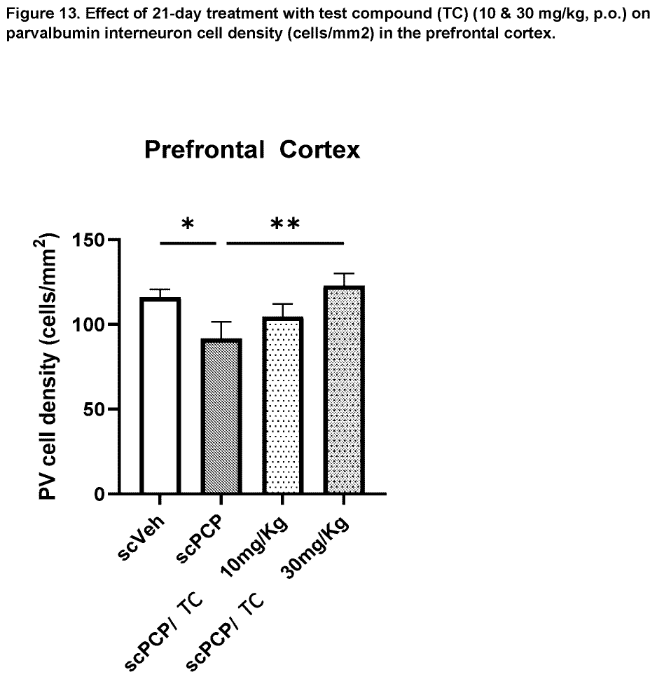

Chronic treatment with the compound of Formula I at 30 mg/kg significantly increased PV cell density in the prefrontal cortex when compared to scPCP group (p<0.01) (FIG. 13).

Effect of 21-Day Treatment with Test Article on Parvalbumin Interneurons in the Dorsal Hippocampus

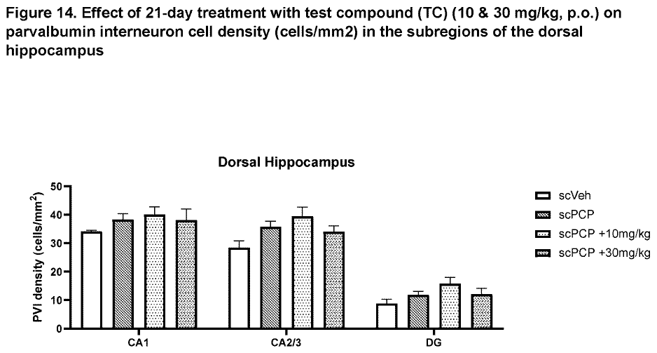

In the dorsal hippocampus there was a significant main effect of treatment and region, with no interaction. Post hoc analysis revealed no significant differences in PV cell density between groups in any region (FIG. 14).

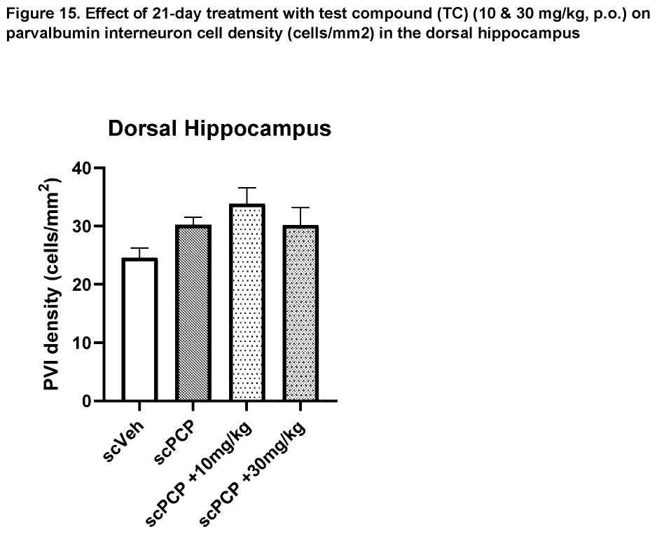

A one-way ANOVA of PV cell density in the total dorsal hippocampus found no difference between the groups (F (3,28)=2.611, p=0.0711) (FIG. 15).

Effect of 21-Day Treatment with Test Article on BDNF Levels in the Prefrontal Cortex

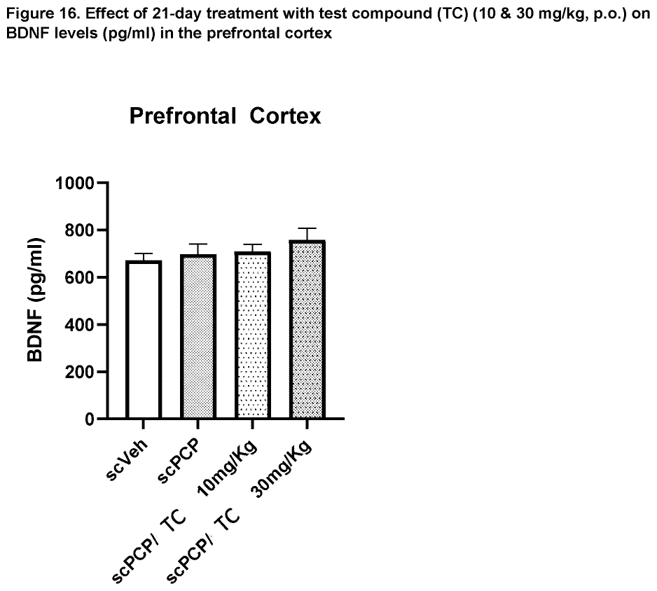

A one-way ANOVA of BDNF levels in the Prefrontal Cortex found no difference between the groups (F (3,27)=0.7652, p=0.5235) (FIG. 16).

Effect of 21-Day Treatment with Test Article on BDNF Levels in the Dorsal Hippocampus

A one-way ANOVA found a trend towards a significant main effect of treatment in the total dorsal hippocampus (F (3,35)=2.360; p=0.0882). Planned post hoc analysis found a reduction in BDNF levels in scPCP animals (p<0.05 vs scVeh). (FIG. 17).

Effect of 21-Day Treatment with Test Article on Synaptic Markers in the Prefrontal Cortex SNAP25:

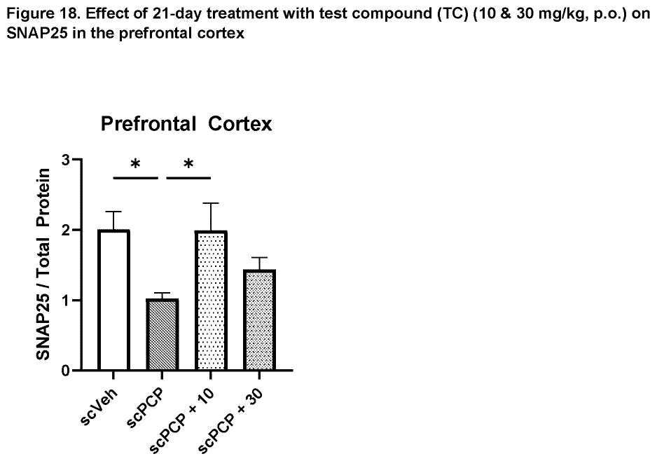

A one-way ANOVA of SNAP25 found a significant main effect of treatment in the total prefrontal cortex (F (3,25)=4.416; p=0.0127). Planned post hoc analysis found a reduction in SNAP25 levels in scPCP animals (p<0.05 vs scVeh). This was increased significantly with chronic OT-003 10 mg/kg treatment (p<0.05) but not OT-003 30 mg/kg treatment (p=0.5412) (FIG. 18).

### PSD95:

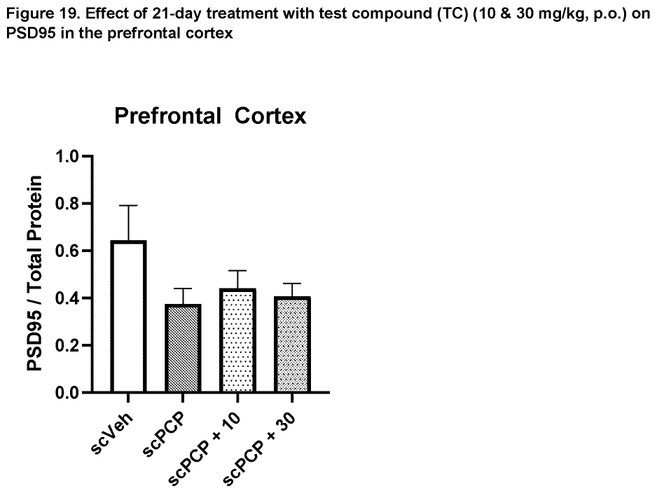

A one-way ANOVA of PSD95 in the total prefrontal cortex found no significant difference between the groups (F (3,28)=1.827, p=0.1651) (FIG. 19).

### Conclusion

This example investigated the effects of 21-day treatment with the compound of Formula I (10 & 30 mg/kg, p.o.) on post-mortem markers (i.e. BDNF, PVIs and synaptic markers) in two different brain regions of relevance to the cognitive and negative symptoms of schizophrenia.

The results of these studies highlight that chronic administration of the compound of Formula I can reverse some of the molecular alterations underlying the behavioural deficits, notably the restoration of PVIs and SNAP25 in the PFC.

Therefore, chronic administration of the compound of Formula I might be effective in the treatment of the cognitive and negative deficits observed in patients with schizophrenia.

### Example 4: Effect of Test Compound to Reverse Sub-Chronic Pcp-Induced Deficits in the Attentional Set-Shifting Task (ASST)

The ability of 21-day treatment with the compound of Formula I, to attenuate the disruption of a complex cognitive task induced by sub-chronic PCP (scPCP) was tested in this example.

The effect of scPCP in attentional set shifting is thought to model cognitive deficit symptoms of schizophrenia, and we have shown that the atypical antipsychotics, clozapine, and risperidone and α7 nicotinic receptor positive allosteric modulators (McLean et al., 2008, 2012) can attenuate the deficit while the typical antipsychotic haloperidol was without effect.

The perceptual attentional set-shifting task investigates the ability of a rat to learn a rule and form an attentional set within the same sorting category (intra-dimensional shift, IDS, as well as the ability to shift attentional set between different sorting categories, extra-dimensional shift, EDS; Birrell and Brown, 2000). It represents a rat analogue of the human CANTAB ID/ED task (Downes et al., 1989) in which schizophrenia patients exhibit impaired set-shifting and reduced cognitive flexibility (Berg et al., 1948; Pantelis et al., 1999; Tyson et al., 2004).

Female rats treated sub-chronically with PCP show a selective deficit in the extra-dimensional shift in this task which is reversed by 7-day treatment with clozapine and risperidone and α7 nicotinic receptor positive modulators, but not haloperidol (McLean et al., 2008, 2012).

### Materials and Methods

### The Set-Shifting Task

### Apparatus:

The test box is a purpose-built cage measuring 59 cm×35 cm×24 cm made of transparent plastic. A fixed 1 cm thick Plexiglas panel permanently divides ⅓ of the length of the box into two separate compartments (choice areas). These two choice areas can be further isolated from each other with the aid of two removable panels. The digging bowls are placed in each choice area, and the rat is given access by lifting the divider(s) which can also be used to deny access to the bowls, such as when an incorrect choice is made.

### Habituation and Training:

Habituation to specific aspects of the testing paradigm commences immediately following the end of the 7-day drug washout period. Habituation to the testing box occurs for 1 h on three consecutive days prior to training. Two smooth ceramic circular digging pots (measuring 9 cm in diameter by 5 cm internal depth) identical to those intended for use during the testing phases are introduced to each home cage. Each bowl is filled with grade 5 sawdust (also covering the home cage floor) and baited with food reward in the form of several ¼ honey nut loops (Kellogg's, UK).

Digging bowls are continuously re-baited during this habituation period and remain in the home cage for the duration of the study.

Following habituation, all rats have to successfully complete the entire training regime in order to proceed to testing. Rats are first trained to dig quickly and reliably in both bowls by progressively covering a single food reward per trial with incrementally thicker layers of digging media. Once a rat has repeatedly demonstrated that it has acquired the training procedure, the dividing panels are introduced. Digging itself is defined as a vigorous movement of front paws to displace digging media and obtain food reinforcement buried 2-2.5 cm below surface level. Failure to dig within 15 minutes will result in termination of the training for that session. Rats will be re-trained the following day.

The second phase of training introduces the concept of simple discrimination (SD) between two media. Rats are presented with identical digging bowls that have two different digging media, only one of which is baited with food reward. Placement of bowls in either the left or right compartment is randomised with the aid of an adapted pseudorandom Gellerman schedule. Rats are permitted to explore both bowls for the first four trials irrespective of which bowl they dug in first, thereby acting as an opportunity to associate food reward with the positive predictor. Subsequent incorrect selections end a trial without the opportunity to explore the correct bowl. A criterion for successful learning of each discrimination is set at 6 consecutive correct trials.

### Testing Protocol:

In all cases rats are tested in the attentional set-shifting procedure 24 h after training. A trial is initiated by raising both dividers to give access to both digging bowls, only one of which is baited. The first stage is the simple discrimination, which is identical to the simple discrimination in the training session on the previous day. Testing continues until the rat reaches a criterion of six consecutive correct responses.

In a test session, rats perform a series of discriminations (for an example see Table 5). For the compound discrimination (CD) a second dimension is introduced (odour), but the correct and incorrect relevant dimension remain the same (digging medium). For the reversals the exemplars and relevant dimensions remain the same (medium or odour), but the rats have to learn that the previously baited dimension is now incorrect and the other dimension is now the correct one. New exemplars are used for the ID and ED shifts. For the ED shift the previously irrelevant dimension (i.e. odour) is now relevant. It has been shown that rats find the difficulty of each dimension change, i.e. medium to odour or odour to medium, equivalent. In simple discrimination, digging medium has been chosen as the relevant parameter for all rats.

TABLE 5
Overall order of discriminations presented to rats
undertaking the attentional set-shifting task

| | Dimensions | | Exemplar combinations | |
|---|---|---|---|---|
| Discriminations | Relevant | Irrelevant | Rewarded | Unrewarded |
| SD | Medium | | M1 | M2 |
| CD | Medium | Odour | M1/O1 | M2/O1 |
| | | | M1/O2 | M2/O2 |
| Rev1 | Medium | Odour | M2/O1 | M1/O1 |
| | | | M2/O2 | M1/O2 |
| IDS | Medium | Odour | M3/O3 | M4/O3 |
| | | | M3/O4 | M4/O4 |
| Rev2 | Medium | Odour | M4/O3 | M3/O3 |
| | | | M4/O4 | M3/O4 |
| EDS | Odour | Medium | O5/M5 | O6/M5 |
| | | | O5/M6 | O6/M6 |
| Rev3 | Odour | Medium | O6/M5 | O5/M5 |
| | | | O6/M6 | O5/M6 |

### In Summary:

In simple discrimination, only one dimension is relevant and introduced (i.e. digging medium; M1/M2).

In compound discrimination, a second dimension is introduced (i.e. odour; O1/O2) while the relevant dimension stays the same (i.e. digging medium).

In reversal 1, the rewarded media is swapped from M1 to M2.
In IDS, new exemplars are introduced (M3/M4/O3/O4) while the relevant dimension stays the same (i.e. digging medium).

In reversal 2, the rewarded media is swapped from M3 to M4.
In EDS, new exemplars are introduced (M5/M6O5/O6) and the relevant dimension is switched from medium to odour.

In reversal 3, the rewarded odour is swapped from O5 to O6.

### Odours and Media Used:

O1: Salted caramel & vanilla.
O2: Mango
O3: Watermelon
O4: White musk
O5: Coconut.
O6: Basil & thyme
M1: Cat litter
M2: Bedding paper
M3 Vermiculite
M4 Wood pellets
M5 Sawdust
M6 Chopped straw.

### ASST Protocol Summary

6 Groups of rats-80 in total were tested in the attentional set shifting task (rats will receive sub-chronic treatment with PCP, 2 mg/kg, i.p. (n=66) or vehicle, i.p. (n=14), twice per day followed by at least 7-days drug free period). This regimen has been found to produce robust and long lasting behavioural and pathological deficits in this paradigm (Neill et al., 2010; Cadinu et al., 2017).

21-Day Treatment with Test Compound

Rats will be tested for their performance in the task following the scPCP treatment regimen and 21-day treatment with 3 doses of test article (3, 10 & 30 mg/kg, p.o.) alone, vehicle (5% DMSO in 0.5 Methylcellulose 400cp) or positive experimental control, risperidone (0.1 mg/kg, i.p.).

### Statistical Analysis

Data will be expressed as mean±s.e.m. (standard error of mean) and analysed by repeated measures GLM (set-shifting phase x treatment group), followed by post hoc analysis in each phase using least significant difference (LSD) test.

### Experimental Groups

| Previous | Reversing the | Dose | | | Dose vol | |
|---|---|---|---|---|---|---|
| treatment | cognitive deficit | (mg/kg) | Route | PTT | (ml/kg) | N |
| 1. scVeh | Vehicle | 0 | PO | 120 | 5 ml/kg | 14 |
| | (5% DMSO in 0.5 | | | min | | |
| | Methylcellulose | | | | | |
| | 400 cp) | | | | | |
| 2. scPCP | Vehicle (5% | 0 | PO | 120 | 5 ml/kg | 14 |
| | DMSO in 0.5 | | | min | | |
| | Methylcellulose | | | | | |
| | 400 cp) | | | | | |
| 3. scPCP | Test article | 3 | PO | 120 | 5 ml/kg | 14 |
| | (21-days) | | | min | | |
| 4. scPCP | Test article | 10 | PO | 120 | 5 ml/kg | 14 |
| | (21-days) | | | min | | |
| 5. scPCP | Test article | 30 | PO | 120 | 5 ml/kg | 14 |
| | (21-days) | | | min | | |
| 6. scPCP | Risperidone | 0.1 | IP | 60 | 1 ml/kg | 10 |
| | (acute) | | | min | | |

### Preparation of Samples Following the ASST Study for Pharmacokinetic Analysis

Plasma and brain samples (groups 1-5) were collected immediately following ASST testing on D21.

Trunk blood was obtained and stored in EDTA coated tubes on ice prior to centrifugation. Blood centrifugation was for 10 min at 5,000 RPM at a temperature of 4° C. Plasma was transferred to Eppendorf vials (2 ml) and stored at −80° C. until analysis. Brains were separated by hemispheres, with one hemisphere (randomly selected) post-fixed in 4% PFA for 24 hours. Samples will then be transferred into 30% sucrose and flash frozen using isopentane prior to storage at −80° C. for immunohistochemistry (IHC) analysis. The other hemisphere was stored directly at −80° C. for protein (e.g. ELISA and westerns) analysis. Three hemispheres (randomly selected) were shipped for PK analysis.

### Results

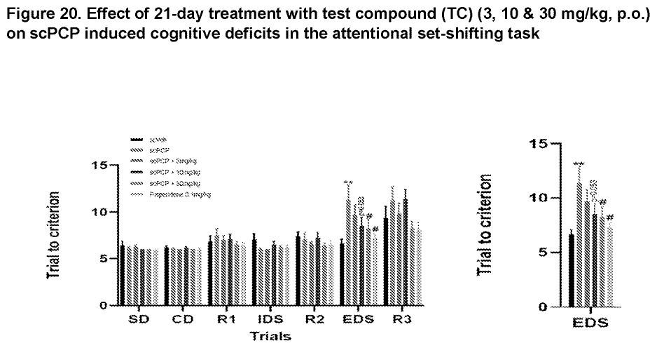

FIG. 20 details the effects of 21-day treatment with the compound of Formula I on scPCP induced cognitive deficits in the attentional set-shifting task. There was a significant (P<0.01) increase in the number of trials to criterion in the extra-dimensional shift (EDS) phase of the attentional set-shifting task following treatment with scPCP compared to vehicle-treated rats.

There were significant reductions (P<0.05) compared to the scPCP group in the number of trials to criterion in the EDS phase following 21-day treatment with the compound of Formula I at 30 mg/kg (P<0.01) and near significant reductions were observed in the 10 mg/kg treated animals (P=0.053).

No significant reduction was observed in 3 mg/kg treated animals.

Risperidone significantly (P<0.01) reversed the scPCP-induced deficit in the EDS phase of the study.

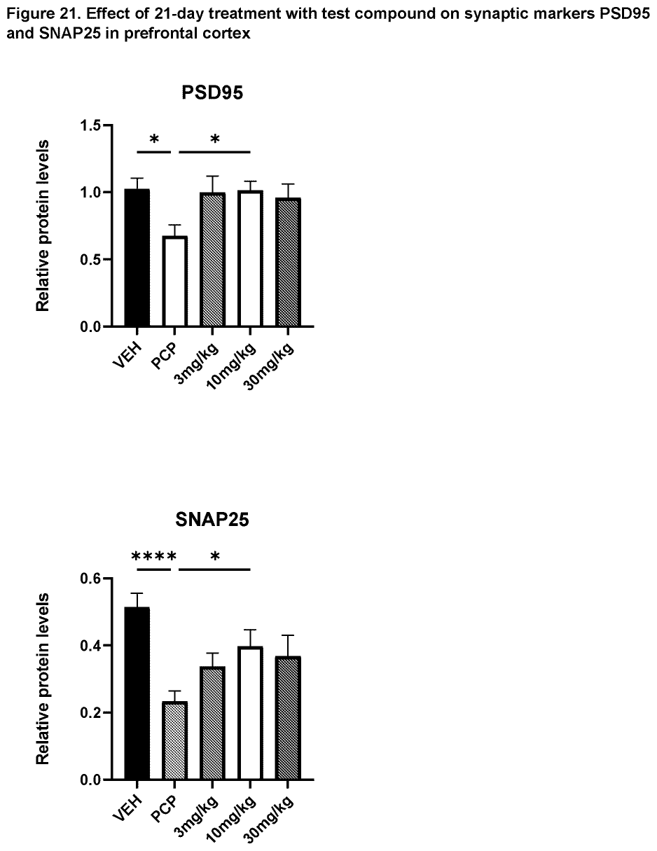

FIG. 21 details the relative protein levels of synaptic markers PSD95 and SNAP25 in animal brains after 21-day treatment with the compound of Formula I. As can be seen the compound of Formula I significantly alleviated scPCP-induced loss of both markers at 3 and 10 mg/kg, but not 30 mg/kg.

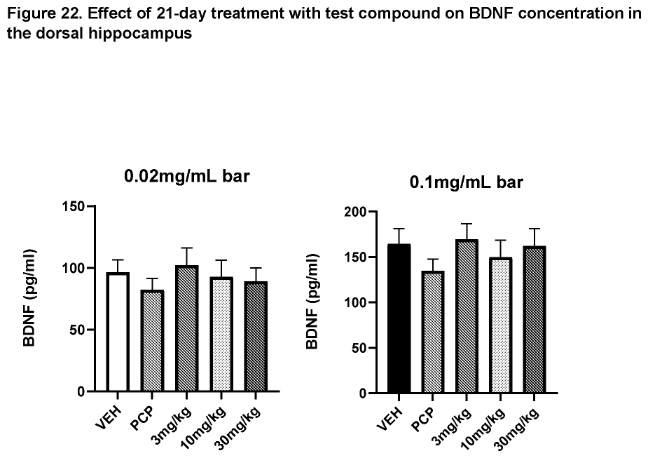

FIG. 22 details the concentration of BDNF in the dorsal hippocampus at two different protein concentrations after 21-day treatment with the compound of Formula I. As can be seen, treatment with scPCP did not significantly reduce the level of BDNF in the rat brains. However, the compound of Formula I was shown to increase the level of BDNF back to that of vehicle treated animals and as such is indicative that the treatment was efficacious at returning the levels to normal.

### Conclusion

The data described in this example demonstrates the ability of the compound of Formula I to reverse the cognitive deficit in the scPCP animals. Such a cognitive deficit is a model of the cognitive symptoms of schizophrenia and as such these data provide evidence that the compound of Formula I may be useful in the treatment of the cognitive symptoms of schizophrenia.

1. A compound of Formula I for use in the treatment of schizophrenia.

2. A compound of Formula I for use according to claim 1, wherein the treatment of schizophrenia is the treatment of one or more symptoms associated with schizophrenia.

3. A compound of Formula I for use according to claim 2, wherein the symptom associated with schizophrenia are cognitive symptoms.

4. A compound of Formula I for use according to claim 2, wherein the symptom associated with schizophrenia are negative symptoms.

5. A compound of Formula I for use according to claim 1, wherein the compound of Formula I is administered with one or more pharmaceutically acceptable excipients.

6. A compound of Formula I for use according to claim 1, wherein the compound of Formula I is formulated in a dosage form selected from a liquid, a lozenge, a fast-disintegrating tablet, a lyophilized preparation, a film, a spray, an aerosol, a sustained-release tablet or capsule, a modified release tablet or capsule, a tablet, a capsule, a cream, an ointment, or a mucoadhesive.

7. A compound of Formula I for use according to claim 1, wherein the compound of Formula I is administered as a single daily dose.

8. A compound of Formula I for use according to claim 1, wherein the compound of Formula I is administered as multiple daily doses.

9. A compound of Formula I for use according to claim 8, wherein the compound of Formula I is administered two, three, four or five times per day.

10. A compound of Formula I for use according to claims 7 to 9, wherein each dose comprises at least 0.001 mg of the compound of Formula I.

11. A compound of Formula I for use according to claims 7 to 9, wherein each dose comprises between about 0.001 mg and about 500 mg of the compound of Formula I.

12. A compound of Formula I for use according to claims 7 to 9, wherein each dose comprises between about 500 mg and about 1000 mg of the compound of Formula I.

13. The compound of Formula I for use according to claim 1, wherein the compound of Formula I is administered with one or more additional drug products.

14. A method of treating schizophrenia in a subject in need thereof, the method comprising administering to the subject a therapeutically effective amount of the compound of Formula I.
